(19) United States
(12) Patent Application Publication
(10) Pub. No.: US 2019/0048086 A1
(43) Pub. Date: Feb. 14, 2019

(54) ANTI-CITRULLINATED HLA POLYPEPTIDE ANTIBODIES AND USES THEREOF

(71) Applicant: Cayman Chemical Company Incorporated, Ann Arbor, MI (US)

(72) Inventor: James Leslies MOBLEY, Brighton, MI (US)

(73) Assignee: Cayman Chemical Company Incorporated, Ann Arbor, MI (US)

(21) Appl. No.: 16/076,599

(22) PCT Filed: Feb. 10, 2017
(86) PCT No.: PCT/US2017/017373

§ 371 Date: Aug. 8, 2018

Related U.S. Application Data
(60) Provisional application No. 62/293,621, filed on Feb. 10, 2016.

(51) Int. Cl. C07K 16/28; A61K 38/08; A61P 19/02; A61K 38/10; C07K 16/44; A61P 29/00

(52) U.S. Cl. CPC C07K 16/2833; A61K 38/08; A61P 19/02; C07K 2317/34; C07K 16/44; A61P 29/00; A61K 38/10

(57) ABSTRACT

The present invention relates to methods for treating an autoimmune disease in a human subject mediated by the presence of a citrullinated epitope QKCitAA, QRCitAA, or RRCitAA in the subject, the method comprising administering a therapeutica1ly effective amount of an antibody or a specific binding fragment thereof that specifically binds to citrullinated epitope QKCitAA, QRCitAA, or RRCitAA. Further aspects of the invention include an antibody or a specific binding fragment thereof, that specifically binds to a human citrullinated epitope QKCitAA, QRCitAA, or RRCitAA.

### CROSS REFERENCE TO RELATED PATENT APPLICATIONS

This PCT International Patent Application claims the benefit of U.S. Provisional Application No. 62/293,621 filed Feb. 10, 2016, the disclosure of which is hereby incorporated by reference into this application in its entirety.

### TECHNICAL FIELD

The present invention relates generally to compositions comprising anti-citrullinated HLA-DR4 antibodies or specific binding fragments thereof, and methods for the treatment of autoimmune diseases in a human subject using same.

### BACKGROUND

MHC class II molecules are a family of cell surface receptors expressed by antigen-presenting cells of the immune system, including monocytes, macrophages, dendritic cells and B lymphocytes (B cells). The function of MHC class II molecules is to display small, potentially antigenic peptides to T lymphocytes (T cells) in a process known as “antigen presentation”. T cells use their T-cell receptors to examine the combination of MHC Class II and peptide to determine if the displayed peptide is a normal component of the host proteome (self) and thus can be ignored, or if the peptide displayed in this context is “foreign” (non-self). Recognition of a foreign peptide displayed by MHC Class II initiates an immune response; the T cell responds by proliferating and producing cytokines, and sends signals back to the antigen presenting cells that a foreign peptide was encountered.

When antigen is present in small amounts, the most efficient antigen presenting cell is a B-cell. Each B-cell expresses a unique high-affinity cell surface receptor that is the surface-bound form of an antibody. Using this high-affinity receptor, B cells are capable of capturing antigens at extremely low concentrations. The captured antigen is engulfed, processed into peptides, and the peptides are made available to MHC class II molecules for display to T cells. If a T-cell recognizes a peptide displayed by a B-cell MHC class II, it instructs the B-cell to proliferate and differentiate into an antibody-producing cell. The antibodies thus produced share the high-affinity antigen binding capacity of the original B-cell surface receptor that captured the intact antigen.

The capacity of an individual MHC class II molecule to bind peptides and display them to T cells is limited; not all possible peptides can be displayed. Therefore, nature has provided each individual with up to six different MHC class II molecules, each capable of binding and displaying slightly different peptides, thereby expanding the breadth of total peptide binding capacity. On a species level, humankind has the genetic capacity to produce dozens of different MHC class II molecules. Because of the tremendous diversity of possible MHC Class II molecules within the species, coupled with the fact that each individual expresses, at most, six different forms, it is quite unlikely that two individuals express the same set of six MHC Class II molecules. This is the basis of tissue transplant rejection. MHC Class II molecules from a tissue donor appear foreign to the T cells of the tissue recipient. This “foreign” recognition event can occur even in in the absence of peptide binding to the donor (allo) MHC Class II molecule; recognition of the empty (no peptide) MHC Class II alone is sufficient to activate a large percentage of T cells in a mismatched recipient (alloreactivity response)(1).

It is generally believed that human rheumatoid arthritis (RA) is an autoimmune disease. It has long been recognized that most patients suffering from RA express one type of MHC class II, HLA-DR4. HLA-DR4 is an HLA-DR serotype; it comprises multiple DRB1*04 gene products that share similar structure. Members of the HLA-DR4 serotype include, but are not limited to DRB1*0401, DRB1*0402, DRB1*0403 DRB1*0404 DRB1*0405 DRB1*0406 DRB1*0407 DRB1*0408 DRB1*0409 DRB1*0410 DRB1*0411 DRB1*0412, and DRB1*0413. Less often, other MHC Class II serotypes (HLA-DR1 and HLA-DR14) rather than HLA-DR4 are expressed in patients with RA. With the advent of DNA sequencing technology, it was discovered that a five amino acid stretch (70-74) in the beta chain of HLA-DR4 (DRB1*0401) is the region of HLA-DR4 most strongly strongly associated with susceptibility to RA. In most cases, the five amino acid sequence is Glutamine-Lysine-Arginine-Alanine-Alanine (QKRAA), as it is in DRB1*0401. In some cases, the amino acid sequence is slightly different, QRRAA (in DRB1*0404, DRB1*0101, and DRB1*0405) or RRRAA (in DRB1*1001). In each case, there is a positively charged amino acid at position 71 (K or R) and a positively charged R at position 72. DRB1*04 alleles that do not share expression of this five amino acid stretch are not associated with RA susceptibility. This “shared epitope” (SE) appears to be a common factor in nearly all human RA patients. In its most basic form, the shared epitope hypothesis proposes that these five common amino acids somehow enable a unique peptide to bind to the MHC class II molecule in RA patients, and that this peptide-MHC combination appears foreign to T cells. The identification of this pro-arthritic peptide has yet to be determined.

Another unique feature of the immune response in RA patients is the production of anti-citrullinated protein antibodies (ACPA). These antibodies are found in ˜70% of RA patients, and are rarely found in other diseases or in healthy individuals. The presence of ACPA has become a reliable diagnostic tool for RA. Citrullination is the enzymatic conversion (deimination) of arginine (R) into another amino acid, citrulline (Cit). Citrullination of proteins or peptides is mediated by peptidylarginine deiminase (PAD) family members, PAD1, PAD2, PAD3, and PAD4. Several naturally-occurring citrullinated proteins have been identified that are bound by ACPA including citrullinated fibrinogen and vimentin. It is currently unknown whether the ACPA are involved in the pathology of RA. The presence of these antibodies has been found in banked plasma samples up to 10 years prior to the onset of disease, suggesting that they are not directly pathogenic.

Multiple genetic association studies have been published looking for genes or genetic mutations associated with RA. By far, the strongest genetic association lies with the HLA-DR4 beta chain (DRB1*0401) or other shared epitope-containing MHC Class II genes. A small number of other genes have been associated in multiple studies, including the gene encoding PAD4 (PADI4). Thus, in RA patients, there is a strong correlation of disease with 1) a specific MHC class II molecule, 2) an enzyme that citrullinates proteins, and 3) the production of anti-citrullinated protein antibodies. These observations led to the generation of a hypothesis that has been the focus of intense research for nearly a decade. The hypothesis proposes that dysregulated PAD4 citrullinates an extracellular protein (eg. fibrinogen or vimentin) that is not normally recognized as foreign by the immune system. The citrullinated protein is captured by B cells expressing surface receptors recognizing citrullinated proteins, ingested, and processed into peptides. Some of the processed peptides contain citrulline residues where they once expressed arginine, and this loss of a charged amino acid allows the modified peptide to bind HLA-DR4 where the unmodified peptide would not have been able to bind. The citrullinated peptide is presented to a T-cell that recognizes this combination as foreign, and directs the antigen-presenting B-cell to produce anti-citrullinated protein antibodies.

Several studies have been published demonstrating that some citrullinated peptides can bind to HLA-DR4 better than the native, uncitrullinated peptide. Other studies have reported that peptides derived from citrullinated vimentin or fibrinogen, when presented by HLA-DR4, can cause T cells to produce cytokines or proliferate slightly more than T cells responding to the unmodified peptides. One lab has reported that mice engineered to express human HLA-DR4 can develop arthritis when immunized with citrullinated human fibrinogen, but other labs have reported an inability to reproduce those findings. Despite much work over many years, no specific peptide has been identified that, when presented by HLA-DR4, can cause arthritis in humans or mice, or produce a robust proliferative response in T cells. Thus, the original peptide presentation hypothesis has not been proven.

In the absence of data that strongly support the peptide-presentation role of the HLA-DR4 shared epitope, alternative hypotheses have been presented. Holoshitz et al. have defined alternative functions of the shared epitope, independent of peptide presentation to T cells. They have reported that the shared epitope can directly bind calreticulin, a potent mediator of innate immunity, and have hypothesized that this interaction is in itself pro-inflammatory, activating signal transduction and nitric oxide generation. This proposed role of the shared epitope is not only independent of peptide presentation to T cells, it is also independent of citrullination, PAD4, and ACAP.

### SUMMARY

In a first aspect, the present invention provides a method for treating an autoimmune disease, for example, rheumatoid arthritis, in a human subject mediated by the presence of HLA-DR4 having at least one citrullinated epitope: QKCitAA, QRCitAA, or RRCitAA in the subject, The method comprises administering a therapeutically effective amount of an antibody or a specific binding fragment thereof that specifically binds to citrullinated epitope QKCitAA, QRCitAA, or RRCitAA to the subject in need thereof

In a second related aspect, the present invention provides an antibody or a specific binding fragment thereof, which specifically binds to a human citrullinated epitope QKCitAA, QRCitAA, or RRCitAA present in the HLA-DR4 receptor protein sequence. The antibody or a specific binding fragment thereof, that specifically binds to a human citrullinated epitope QKCitAA, QRCitAA, or RRCitAA does not substantially bind to QKRAA, QRRAA or RRRAA peptide sequences present in the HLA-DR4 receptor protein sequence.

### BRIEF DESCRIPTION OF THE DRAWINGS

FIG. 1 depicts a schematic representation of the proposed mechanisms for presentation of a citrullinated shared epitope from HLA-DR4 receptors on B cells to activate T cells for generation of autoimmune responses in RA patients.

FIG. 2A depicts ELISA binding results of isolated anti MHC class II peptide (EQKCitAA) monoclonal antibody against various antigens: MHC class II peptide (EQKRAA), citrullinated MHC class II peptide (EQKCitAA), enolase, citrullinated enolase, H3 protein and citrullinated H3 protein.

FIG. 2B depicts ELISA binding results of isolated anti MHC class II peptide (EQKCitAA) monoclonal antibody against various antigens MHC class II peptide (EQKRAA), citrullinated MHC class II peptide (EQKCitAA), H3 peptide (1-21) and citrullinated H3 peptide (Cit R 2+8+17).

### DETAILED DESCRIPTION

### Definitions:

For purposes of this disclosure, unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). These references are hereby incorporated into this disclosure by reference in their entireties.

All publications and references mentioned herein are incorporated by reference. Nothing herein is to be construed as an admission that the invention is not entitled to antedate such disclosure by virtue of prior invention.

It must be noted that, as used herein, and in the appended claims, the singular forms “a”, “an”, and “the” include plural reference unless the context clearly dictates otherwise.

Embodiments including the transition phrase “consisting of” or “consisting essentially of” include only the recited components and inactive ingredients.

As used herein, the term “about” means plus or minus 10% of the numerical value of the number with which it is being used. Therefore, about 50% means in the range of 45%-55%.

“Optional” or “optionally” may be taken to mean that the subsequently described structure, event or circumstance may or may not occur, and that the description includes both instances where the event occurs and instances where it does not.

The term “epitope” as used herein refers to the collective features of a molecule, such as primary structure, and charge, that together form a region on an antigen at which an antibody binds, by virtue of the antibody's antigen-binding site (called the paratope). An epitope can be either defined as a set of amino acid residues that are close together in the primary sequence of the protein, or of amino acid residues which are well separated in the primary sequence, but are brought together as a result of the natural folding of the protein to its native, fully functional shape. Epitopes consisting of residues close together in the primary sequence are called contiguous, continuous, sequential or linear epitopes, whereas epitopes consisting of residues separated in the primary sequence are by contrast called discontinuous, conformational or “assembled” epitopes.

Epitopes are present in nature, and can be mapped, isolated, purified or otherwise prepared/derived by humans. For example, epitopes can be prepared by isolation from a natural resource, or they can be synthesized in accordance with standard protocols in the art. One of these methods is the use of synthetic fragments (peptides) of the protein antigen, which can be similar enough to the homologous parts of the whole antigen to permit binding by the antibody. The affinity of the antibody for the epitope must be such that the antibody/peptide complex does not dissociate significantly under the conditions of an immunoassay. This situation occurs with linear epitopes, thus allowing the use of peptides to define those epitopes, and the use of amino acids and amino acid mimics to define individual epitopes. A derived/prepared epitope can be an analog of a native epitope. Throughout this disclosure, the terms epitope and hapten are often used interchangeably.

The term “specific binding molecule” is used herein to indicate a molecule, preferably a small molecule, capable of specific binding. Specific binding in this respect is intended to mean that the molecule is capable of binding to a selected target molecule whereas it will not bind to another, non-related target molecule under the same conditions. For instance, a binding molecule is said to specifically bind to serum albumin when it binds to serum albumin and binds less or not at all to another or preferably any other protein found in serum. Preferred specific binding molecules as used herein in the present invention include specific bindng agents, for example, an antibody or an antibody fragment thereof, that binds to a citrullinated epitope QKCitAA, or a citrullinated epitope QRCitAA, or it can bind to the citrullinated epitope RRCitAA.

The term “specifically reacts with a citrullinated epitope” or “reactive with a citrullinated epitope” or “reactive with a citrulline epitope” in this context means that the specific binding molecule or antibody reacts with a structure such as a peptide containing a citrulline residue, for example, citrullinated epitope QKCitAA, or citrullinated epitope QRCitAA, or citrullinated epitope RRCitAA, whereas the antibody, or antibody fragment thereof reacts less or preferably not at all with the same structure containing an arginine residue instead of the citrulline residue. The term “peptide” should be interpreted as a structure that is capable of presenting the citrulline residue in the correct context for immunoreactivity with the specific binding molecules as described herein, preferably in the same context as it appears in the human or animal body, preferably in the context of a native polypeptide.

The “specific binding molecule” may be a molecule, preferably a small molecule, composed of DNA, RNA, peptide, protein domain, whole proteins, or combinations thereof or parts thereof, that are capable of specifically binding to a target compound. Preferred examples of specific binding molecules are peptides or antibodies or antibody binding fragments thereof

Native antibodies (also known as immunoglobulins) are gamma globulin proteins that may be found in blood or other bodily fluids of vertebrates, and are used by the immune system to identify and neutralize foreign objects, such as bacteria and viruses.

Native antibodies are typically made of basic structural units--each with two large heavy chains and two small light chains, for example, monomers with one unit, dimers with two units or pentamers with five units. Antibodies are produced by a white blood cell called a B cell. There are several different types of heavy chains, resulting in different kinds of antibodies. Antibodies may be grouped into different isotypes based on which heavy chain they possess. Five different antibody isotypes are known in mammals that perform different roles, and help direct the appropriate immune response for each different type of foreign object they encounter. Some animal species such as Camelids (e.g., llamas) and sharks may have aberrant antibody structures.

Although the general structure of all antibodies is very similar, a small region at the tip of the protein is extremely variable, allowing millions of antibodies with slightly different tip structures to exist. This region is known as the hypervariable region. Each of these variants can bind to a different target, known as an antigen. This huge diversity of antibodies allows the immune system to recognize an equally wide diversity of antigens.

The unique part of the antigen recognized by an antibody is called an epitope. These epitopes bind with their antibody in a highly specific interaction that allows antibodies to identify and bind only their unique antigen in the midst of the millions of different molecules that make up an organism. Recognition of an antigen by an antibody tags it for attack by other parts of the immune system. Antibodies can also neutralize targets directly, for example, by binding to a part of a pathogen that it needs to cause an infection.

The large and diverse population of antibodies is generated by random combinations of a set of gene segments that encode different antigen-binding sites (or paratopes), followed by random mutations in this area of the antibody gene, which create further diversity. Antibody genes also re-organize in a process called class switching that changes the base of the heavy chain to another, creating a different isotype of the antibody that retains the antigen-specific variable region. This allows a single antibody to be used in several different isotypes by several different parts of the immune system.

Wild-type antibodies are typically composed of two identical pairs of polypeptide chains, each pair having one light chain and one heavy chain. Each of the heavy and light chains is made up of two distinct regions, referred to as the variable and constant regions. Thus there is the variable heavy chain (VH), the constant heavy chain (CH), the variable light chain (VL) and the constant light chain (CL). The variable regions (VH and VL) of an antibody contains the antigen binding sequences of the molecule and thus determine the specificity of an antibody for its target antigen. In the variable region, three loops for each of the variable domains of the heavy chain and light chain forms the antigen-binding site. Each of the three loops is referred to as a complementary-determining region, or “CDR”. There are six CDRs, three per heavy chain and three per light chain, designated VHCDR1, VHCDR2, VHCDR3, VLCDR1, VLCDR2 and VLCDR3. The variable region outside, and in between, the CDRs is referred to as the framework region.

An antibody may be selected from the group consisting of single-chain antibodies, single-chain variable fragments (scFvs), fragment antigen-binding regions (Fabs), recombinant antibodies, monoclonal antibodies, fusion proteins comprising the antigen-binding domain of a native antibody or an aptamer, single-domain antibodies (sdabs), also known as VHH antibodies, nanobodies (camelid-derived single-domain antibodies), shark IgNAR-derived single-domain antibody fragments called VNAR, anticalins, aptamers (DNA or RNA) and active components or fragments thereof

In another embodiment, an antibody is a fusion protein comprising the antigen-binding domain of a native antibody or an aptamer, such as an aptamer in the form of DNA or RNA.

The term “or part thereof” or “specific binding fragments thereof” in the context of an antibody or other specific binding molecule is meant to refer to the part of the antibody or specific binding molecule that makes up the specific binding site of the antibody or specific binding molecule and may be interpreted as the part of an antibody or specific binding molecule that is still capable of reacting with the same epitope as the entire antibody or specific binding molecule.

Human antibodies or specific binding fragments thereof are a preferred embodiment of the disclosure. Preferably, IgG1 (e.g., IgG1) antibodies having an IgG1 heavy chain and a lambda or kappa light chain may be used advantageously. However, other human antibody isotypes are also encompassed by the disclosure, including IgG2, IgG3, IgG4, IgM, IgAl, IgA2, IgAsec, IgD and IgE in combination with a kappa or lambda light chain. Also, all animal-derived antibodies of various isotypes can be used in the disclosure. The antibodies can be full-size antibodies or antigen-binding fragments of antibodies, including Fab, F(ab′)2, single-chain Fv fragments, or single-domain VHH, VH or VL single domains.

The term “specific binding molecules reactive with a citrullinated HLA-DR4 epitope” is to be interpreted as specific binding molecules that specifically react with a citrulline residue in the context of a larger structure such as HLA-DR4 polypeptide or portion thereof.

“Isolated” refers to the state in which antibodies, nucleic acids encoding such antibodies and host cells according to the invention will preferably be in. With respect to antibodies and nucleic acid, “isolated” means that antibodies and nucleic acids will generally be free or substantially free of material with which they are naturally associated such as other polypeptides or nucleic acids with which they are found in their natural environment or in the environment in which they are prepared, (e.g. cell culture) for example when such preparations is by recombinant DNA technology. When applied to host cells, “isolated” refers to host cells isolated from the organism from where they originate, such as, for example, cells in cell culture. Antibodies, nucleic acids and host cells may be formulated with diluents or adjuvant and still for practical purposes be isolated.

“Amino acid modification” refers to amino acid residue substitutions, insertions and deletions in a polypeptide sequence. “Substitution” refers to the replacement of an amino acid residue at a particular position in a polypeptide sequence with another amino acid residue. “Insertion” refers to the addition of an amino acid residue between two preexisting amino acid residues a particular position in a polypeptide sequence. “Deletion” refers to removal of an amino acid residue at a particular position in a polypeptide sequence.

In various embodiments, the present disclosure provides a novel role for the shared epitope that does not require presentation of a citrullinated peptide by HLA-DR4, but is associated with PAD4 and ACAP.

A. Anti-Citrullinated Shared Epitope Antibodies

The present disclosure identifies a novel role for the shared epitope that does not require presentation of a citrullinated peptide by HLA-DR4, but is associated with PAD4 and ACAP. It takes advantage of the following observations: 1) PAD4 is autocitrullinated, 2) a subset of RA patients make antibodies that recognize PAD4, 3) the shared epitope sequence QKRAA contains an arginine residue that can be citrullinated. The present disclosure proposes that B cells with cell surface receptors capable of recognizing citrullinated PAD4, uncitrullinated PAD4, or a protein in the process of being citrullinated by PAD4 can directly or indirectly capture PAD4 on the cell surface. The captured PAD4 remains enzymatically active and citrullinates adjacent HLA-DR4 molecules at the shared epitope QKRAA, QRRAA, or RRRAA, either on the B cell surface or in within the phagosome. The arginine at position 72 of the shared epitope is one of the citrullinated residues. Thus modified, the citrullinated HLA-DR4 appears foreign to T cells in an alloreactive manner. Alternatively, the citrullinated HLA-DR4 can present unique peptides to T cells that would not be presented if the HLA-DR4 had not been citrullinated. In either situation, the responding T cells would instruct the B cells to produce antibodies with the same binding specificity as the surface receptors, including anti-PAD4, anti-citrullinated PAD4, or other citrullinated proteins.

Recognition of this novel mechanism informs the development of new pharmacologic agents for the treatment or prevention of RA including but not limited to cytolytic therapeutic antibodies, shared epitope binding fragments of antibodies, or antibody-like biologic molecules that bind the citrullinated shared epitope and destroy the cells expressing the citrullinated MHC Class II molecule and prevents T cell activation.

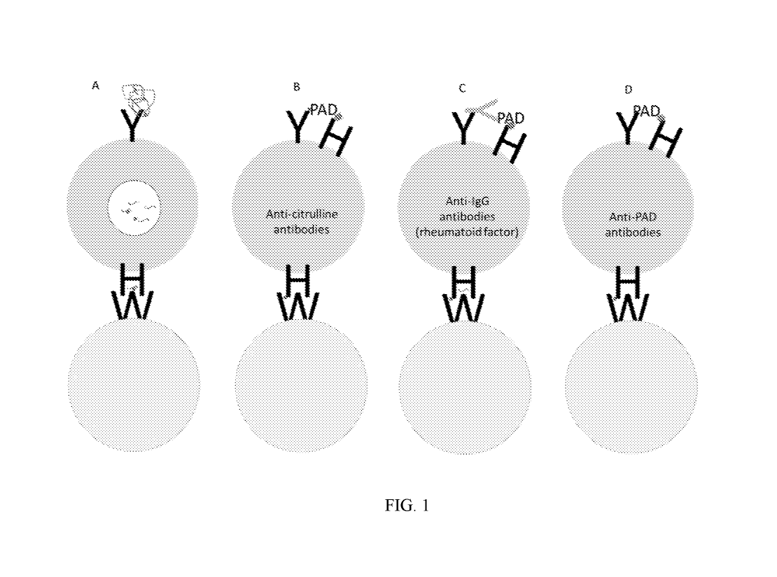

As shown in FIG. 1, parts B-D depict the direct citrullination of a shared epitope QKRAA to QKCitAA. Although not shown, the shared epitope could also include QRRAA, or RRRAA which may be citrullinated by PAD to generate the T cell reactive epitopes QRCitAA, and RRCitAA. In exemplary embodiments as shown in FIG. 1, part B, a B-cell surface immunoglobulin (Y) specific for citrullinated proteins binds to autocitrullinated PAD. The PAD enzyme citrullinates the shared epitope QKRAA to QKCitAA on a nearby HLA-DR4 molecule (the small circle on the HLA-DR4 (H). The citrullinated HLA-DR4 molecule is directly recognized by a T cell receptor (W) with or without additional bound peptide. The T cell instructs a B cell to produce anti-citrullinated protein antibodies (ACAP).

In FIG. 1, part C, a B cell surface immunoglobulin (Y) specific for IgG, binds an antibody that has bound PAD. The bound PAD citrullinates the shared epitope sequence on a nearby HLA-DR4 molecule (H). The citrullinated HLA-DR4 molecule is directly recognized by a T cell receptor (W) with or without additional bound peptide. The T cell instructs the B-cell to produce anti-IgG antibodies (rheumatoid factor).

In FIG. 1, part D, a B cell surface immunoglobulin (Y) specific for PAD binds PAD. The B cell bound PAD citrullinates the shared epitope QKRAA in the HLA-DR4 (H) to QKCitAA amino acid sequence on a nearby HLA-DR4 molecule (H). The citrullinated HLA-DR4 molecule is directly recognized by a T cell receptor (W) with or without additional bound peptide. The T cell instructs the B cell to produce anti-PAD antibodies. As used herein, an exemplary PAD can include PAD4 or PAD2 or combinations thereof

Without wishing to be bound to any specific theory, it is believed that B cells with cell surface receptors capable of recognizing or binding citrullinated PAD4, uncitrullinated PAD4, or a protein in the process of being citrullinated by PAD4 can directly or indirectly capture PAD4 on the cell surface of the B cell. The captured PAD4 remains enzymatically active and citrullinates adjacent HLA-DR4 molecules, either on the B cell surface or within the B cell phagosome. The arginine at position 72 of the shared epitope of HLA-DR4 is one of the citrullinated residues. Thus modified, the citrullinated HLA-DR4 receptor appears foreign to T cells in an alloreactive manner. Alternatively, the citrullinated HLA-DR4 can present unique peptides to T cells that would not be presented to T cells if the HLA-DR4 had not been citrullinated. In either situation, the responding T cells would instruct the B cells to produce antibodies with the same binding specificity as the surface receptors, including PAD4, citrullinated PAD4, or other citrullinated proteins, in addition to becoming activated and release pro-inflammatory cytokines such as TNF-a.

Recognition of this novel mechanism informs the development of new pharmacologic agents for the treatment or prevention of RA, including but not limited to, cytolytic therapeutic antibodies, antigen-binding fragments thereof, or antibody-like biologic molecules that bind the citrullinated shared epitope QKCitAA, QRCitAA, or RRCitAA and destroy the cells expressing the citrulllnated MHC Class II molecule. Binding of antibodies or antibody-like biologic molecules to the citrullinated shared epitope present on antigen presenting cells, for example, B cells enables elimination of these cells via antibody-dependent cellular toxicity (ADCC) mechanisms, and complement-dependent cytotoxicity (CDC) among other mechanisms.

In some embodiments, the present invention provides useful therapeutic compositions comprising mammalian antibodies that are specific for, and bind with high affinity to the citrullinated epitope QKCitAA, or citrullinated epitope QRCitAA, or citrullinated epitope RRCitAA. As used herein, the amino acid sequence referred to as the citrullinated shared epitopes, wherein the pre-citrullinated epitope is QKRAA, QRRAA, or RRRAA and one of the arginine residues in the pre-citrullinated epitope is acted upon by the enzyme peptidylarginine deiminase (PAD) by deiminating arginine into citrulline (“Cit”).

Peptidylarginine deiminase (PAD; EC 3.5.3.15) enzymes catalyze the conversion of arginine residues to citrulline residues in proteins. No tRNA exists for citrulline; the presence of citrulline residues in proteins is exclusively the result of post-translational modification. In mammals (humans, mice and rats), five PAD isotypes (PAD1-PAD6; “PAD4” and “PADS” are used for the same isotype), each encoded by a distinct gene, have been identified (Vossenaar et al., Bioessays 25, 1106-1118, 2003). All these enzymes rely strongly on the presence of Ca²⁺ for activity and are unable to convert free L-arginine into free L-citrulline. Free L-arginine can be converted to free L-citrulline by nitric oxide synthase (EC 1.14.13.39) in eukaryotes or by arginine deiminase (EC 3.5.3.6) in bacteria. These enzymes are not Ca²⁺ dependent.

Methods for making mammalian antibodies or fragments thereof, that are specific for, and bind with high affinity to the citrullinated epitope QKCitAA, or citrullinated epitope QRCitAA, or citrullinated epitope RRCitAA are known in the art. In some embodiments, cell fusion methods for making monoclonal antibodies may be used in the methods of the invention such as those disclosed in U.S. Pat. No. 5,916,771, incorporated herein by reference in its entirety. Briefly, according to this method, DNA encoding the desired heavy chain (or a fragment of the heavy chain) is introduced into a first mammalian host cell, while DNA encoding the desired light chain (or a fragment of the light chain) is introduced into a second mammalian host cell. The first transformed host cell and the second transformed host cell are then combined by cell fusion to form a third cell. Prior to fusion of the first and second cells, the transformed cells may be selected for specifically desired characteristics, e.g., high levels of expression. After fusion, the resulting hybrid cell contains and expresses both the DNA encoding the desired heavy chain and the DNA encoding the desired light chain, resulting in production of the multimeric antibody. In certain embodiments, monoclonal antibodies are produced by standard techniques. In certain embodiments, monoclonal antibodies are produced by hybridoma-based methods which are well known in the art. In various embodiments, methods for producing mouse monoclonal antibodies directed to the epitope QKCitAA from sequences of HLA-DR4 are known to those skilled in the art, for example, in certain such embodiments, a suitable animal, such as a mouse, rat, hamster, monkey, or other mammal, is immunized with an immunogen to produce antibody-secreting cells. In various embodiments, the immunogen can be the citrullinated epitope QKCitAA, or citrullinated epitope QRCitAA, or citrullinated epitope RRCitAA, or it can be one of the citrullinated epitopes linked or fused to a polypeptide or protein. In these embodiments, the screening process would yield hybridomas expressing monoclonal antibodies that bound to the citrullinated epitope QKCitAA, but not the conjugated polypeptide or protein.

In certain embodiments, the antibody-secreting cells are B cells, such as lymphocytes or splenocytes. In certain embodiments, lymphocytes (e.g., human lymphocytes) are immunized in vitro to generate antibody-secreting cells. See, e.g., Borreback et al. (1988) Proc. Nat'l Acad. Sci. USA 85:3995-3999.

In certain embodiments, antibody secreting cells are fused with an “immortalized” cell line, such as a myeloid-type cell line, to produce hybridoma cells. In certain embodiments, hybridoma cells that produce the desired antibodies are identified, for example, by ELISA. In certain embodiments, such cells can then be subcloned and cultured using standard methods. In certain embodiments, such cells can also be grown in vivo as ascites tumors in a suitable animal host. In certain embodiments, monoclonal antibodies are isolated from hybridoma culture medium, serum, or ascites fluid using standard separation procedures, such as affinity chromatography. Guidance for the production of hybridomas and the purification of monoclonal antibodies according to certain embodiments is provided, for example, in Harlow and Lane (1988) Antibodies: A Laboratory Manual Ch. 8 (Cold Spring Harbor Laboratory, Cold Spring Harbor, N.Y.).

In certain embodiments, mouse monoclonal antibodies are produced by immunizing genetically altered mice with an immunogen. In certain such embodiments, the mice are HLA-DR4-deficient mice. In certain such embodiments, the mice are “knockout” mice that lack all or part of a gene encoding human HLA-DR4. In certain embodiments, such knockout mice are immunized with the citrullinated epitope QKCitAA, or citrullinated epitope QRCitAA, or citrullinated epitope RRCitAA, either alone or when conjugated to a polypeptide or protein.

In certain embodiments, human monoclonal antibodies are raised in transgenic animals (e.g., mice) that are capable of producing human antibodies. See, e.g., U.S. Pat. Nos. 6,075,181 A and 6,114,598 A; and WO 98/24893 A2. For example, in certain embodiments, human immunoglobulin genes are introduced (e.g., using yeast artificial chromosomes, human chromosome fragments, or germline integration) into mice in which the endogenous Ig genes have been inactivated. See, e.g., Jakobovits et al. (1993) Nature 362:255-258; Tomizuka et al. (2000) Proc. Nat'l Acad. Sci. USA 97:722-727; and Mendez et al. (1997) Nat. Genet. 15:146-156 (describing the XenoMouse II.RTM. line of transgenic mice).

In certain embodiments, such transgenic mice are immunized with an immunogen. In certain such embodiments, lymphatic cells (such as B cells) from mice that express antibodies are obtained. In certain such embodiments, such recovered cells are fused with an “immortalized” cell line, such as a myeloid-type cell line, to produce hybridoma cells. In certain such embodiments, hybridoma cells are screened and selected to identify those that produce antibodies specific to the antigen of interest. Certain exemplary methods and transgenic mice suitable for the production of human monoclonal antibodies are described, e.g., in Green (1999) J. Immunol. Methods 231:11-23; and WO 98/24893. In certain embodiments, human monoclonal antibodies against the citrullinated epitope QKCitAA, or citrullinated epitope QRCitAA, or citrullinated epitope RRCitAA are suitable for use as therapeutic antibodies or fragments thereof for the treatment of an autoimmune disease, for example, rheumatoid arthritis.

Phage and Yeast Display-Based Methods

In certain embodiments, human monoclonal antibodies are produced using a display-based method, such as, for example, any of those described below.

In certain embodiments, a monoclonal antibody is produced using phage display techniques. Certain exemplary antibody phage display methods are known to those skilled in the art and are described, for example, in Hoogenboom, Overview of Antibody Phage-Display Technology and Its Applications, from Methods in Molecular Biology: Antibody Phage Display: Methods and Protocols (2002) 178:1-37 (O'Brien and Aitken, eds., Human Press, Totowa, N.J.). For example, in certain embodiments, a library of antibodies are displayed on the surface of a filamentous phage, such as the nonlytic filamentous phage fd or M13. In certain embodiments, the antibodies are antibody fragments, such as scFvs, Fabs, Fvs with an engineered intermolecular disulfide bond to stabilize the VH-VL pair, and diabodies. In certain embodiments, antibodies with the desired binding specificity can then be selected. Certain exemplary embodiments of antibody phage display methods are described in further detail below.

In certain embodiments, an antibody phage-display library can be prepared using certain methods known to those skilled in the art. See, e.g., Hoogenboom, Overview of Antibody Phage-Display Technology and Its Applications, from Methods in Molecular Biology: Antibody Phage Display: Methods and Protocols (2002) 178:1-37 (O'Brien and Aitken, eds., Human Press, Totowa, N.J.). In certain embodiments, variable gene repertoires are prepared by PCR amplification of genomic DNA or cDNA derived from the mRNA of antibody-secreting cells. For example, in certain embodiments, cDNA is prepared from mRNA of B cells expressing antibodies against citrullinated epitope QKCitAA. In certain embodiments, cDNA encoding the variable regions of heavy and light chains is amplified, for example, by PCR.

In certain embodiments, heavy chain cDNA and light chain cDNA are cloned into a suitable vector. In certain embodiments, heavy chain cDNA and light chain cDNA are randomly combined during the cloning process, thereby resulting in the assembly of a cDNA library encoding diverse scFvs or Fabs. In certain embodiments, heavy chain cDNA and light chain cDNA are ligated before being cloned into a suitable vector. In certain embodiments, heavy chain cDNA and light chain cDNA are ligated by stepwise cloning into a suitable vector.

In certain embodiments, cDNA is cloned into a phage display vector, such as a phagemid vector. Certain exemplary phagemid vectors, such as pCES1, are known to those skilled in the art. In certain embodiments, cDNA encoding both heavy and light chains is present on the same vector. For example, in certain embodiments, cDNA encoding scFvs are cloned in frame with all or a portion of gene III, which encodes the minor phage coat protein pill. In certain such embodiments, the phagemid directs the expression of the scFv-pIII fusion on the phage surface. Alternatively, in certain embodiments, cDNA encoding heavy chain (or light chain) is cloned in frame with all or a portion of gene III, and cDNA encoding light chain (or heavy chain) is cloned downstream of a signal sequence in the same vector. The signal sequence directs expression of the light chain (or heavy chain) into the periplasm of the host cell, where the heavy and light chains assemble into Fab fragments. Alternatively, in certain embodiments, cDNA encoding heavy chain and cDNA encoding light chain are present on separate vectors. In certain such embodiments, heavy chain and light chain cDNA is cloned separately, one into a phagemid and the other into a phage vector, which both contain signals for in vivo recombination in the host cell. Recombinant phagemid or phage vectors are introduced into a suitable bacterial host, such as E. coli. In certain embodiments using phagemid, the host is infected with helper phage to supply phage structural proteins, thereby allowing expression of phage particles carrying the antibody-pill fusion protein on the phage surface.

In various exemplary embodiments, “synthetic” antibody libraries are constructed using repertoires of variable genes that are rearranged in vitro. For example, in certain embodiments, individual gene segments encoding heavy or light chains (V-D-J or V-J, respectively) are randomly combined using PCR. In certain such embodiments, additional sequence diversity can be introduced into the CDRs, and possibly FRs, e.g., by error prone PCR. In certain such embodiments, additional sequence diversity is introduced into CDR3, e.g., H3 of the heavy chain.

In certain embodiments, “naive” or “universal” phage display libraries are constructed as described above using nucleic acid from an unimmunized animal. In certain embodiments, the unimmunized animal is a human. In certain embodiments, “immunized” phage display libraries are constructed as described above using nucleic acid from an immunized animal. In certain embodiments, the immunized animal is a human, rat, mouse, hamster, or monkey. In certain such embodiments, the animals are immunized with any of the immunogens described below.

In certain embodiments, the selection of antibodies having the desired binding specificity from a phage display library is achieved by successive panning steps. In certain embodiments of panning, library phage preparations are exposed to antigen. In certain such embodiments, the phage-antigen complexes are washed, and unbound phage are discarded. In certain such embodiments, bound phage are recovered and subsequently amplified by infecting E. coli. In certain such embodiments, monoclonal antibody-producing phage may be cloned by picking single plaques. In certain embodiments, the above process is repeated.

In certain embodiments, the antigen used in panning is any of the immunogens described below. In certain embodiments, the antigen is immobilized on a solid support to allow purification of antigen-binding phage by affinity chromatography. In certain embodiments, the antigen is biotinylated, thereby allowing the separation of bound phage from unbound phage using streptavidin-coated magnetic beads. In certain embodiments, the antigen may be immobilized on cells (for direct panning), in tissue cryosections, or on membranes (e.g., nylon or nitrocellulose membranes). Other variations of certain panning procedures may be routinely determined by one skilled in the art.

In certain embodiments, a yeast display system is used to produce monoclonal antibodies. In certain such systems, an antibody is expressed as a fusion protein with all or a portion of the yeast AGA2 protein, which becomes displayed on the surface of the yeast cell wall. In certain such embodiments, yeast cells expressing antibodies with the desired binding specificity can then be identified by exposing the cells to fluorescently labeled antigen. In certain such embodiments, yeast cells that bind the antigen can then be isolated by flow cytometry. See, e.g., Boder et al. (1997) Nat. Biotechnol. 15:553-557.

In certain embodiments, a monoclonal antibody is produced by recombinant techniques. See, e.g., U.S. Pat. No. 4,816,567. In certain such embodiments, nucleic acid encoding monoclonal antibody chains are cloned and expressed in a suitable host cell. For example, in certain embodiments, RNA can be prepared from cells expressing the desired antibody, such as mature B cells or hybridoma cells, using standard methods. In certain embodiments, the RNA can then be used to make cDNA using standard methods. In certain embodiments, cDNA encoding a heavy or light chain polypeptide is amplified, for example, by PCR, using specific oligonucleotide primers. In certain embodiments, the cDNA is cloned into a suitable expression vector. In certain embodiments, the expression vector is then transformed or transfected into a suitable host cell, such as a host cell that does not endogenously produce antibody. Certain exemplary host cells include, but are not limited to, E. coli, COS cells, Chinese hamster ovary (CHO) cells, and myeloma cells. In certain embodiments, wherein heavy and light chains are coexpressed in the same host, reconstituted antibody may be isolated.

In certain embodiments, cDNA encoding a heavy or light chain can be modified. For example, in certain embodiments, the constant region of a mouse heavy or light chain can be replaced with the constant region of a human heavy or light chain. In this manner, in certain embodiments, a chimeric antibody can be produced which possesses human antibody constant regions but retains the binding specificity of a mouse antibody.

In certain embodiments, recombinant antibodies can be expressed in certain cell lines. In certain embodiments, sequences encoding particular antibodies can be used for transformation of a suitable mammalian host cell. According to certain embodiments, transformation can be by any known method for introducing polynucleotides into a host cell. Certain exemplary methods include, but are not limited to, packaging the polynucleotide in a virus (or into a viral vector) and transducing a host cell with the virus (or vector) and using certain transfection procedures known in the art, as exemplified by U.S. Pat. Nos. 4,399,216, 4,912,040, 4,740,461, and 4,959,455. In certain embodiments, the transformation procedure used may depend upon the host to be transformed. Certain exemplary methods for introduction of heterologous polynucleotides into mammalian cells are known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Certain exemplary mammalian cell lines available as hosts for expression are known in the art and include, but are not limited to, many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g., Hep G2), and a number of other cell lines. In certain embodiments, cell lines may be selected by determining which cell lines produce high levels of antibodies that specifically bind citrullinated epitope QKCitAA, or citrullinated epitope QRCitAA, or citrullinated epitope RRCitAA.

In various embodiments, the antibodies that bind to a citrullinated HLA-DR4 epitope are humanized antibodies. As used herein, the term “humanized antibody” refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences.

A “humanized antibody” is an antibody comprising a humanized light chain and a humanized heavy chain immunoglobulin. For example, a humanized antibody would not encompass a typical chimeric antibody, because, e.g., the entire variable region of a chimeric antibody is non-human. One says that the donor antibody has been “humanized”, by the process of “humanization”, because the resultant humanized antibody is expected to bind to the same antigen as the donor antibody that provides the CDR's. For the most part, humanized antibodies are human immunoglobulins (recipient antibody) in which hypervariable region residues of the recipient are replaced by hypervariable region residues from a non-human species (donor antibody) such as mouse, rat, rabbit or a non-human primate having the desired specificity, affinity, and capacity. In some instances, Framework Region (FR) residues of the human immunoglobulin are replaced by corresponding non-human residues.

Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin that immunospecifically binds to a FcyRIIB polypeptide, that has been altered by the introduction of amino acid residue substitutions, deletions or additions (i.e., mutations). In some embodiments, a humanized antibody is a derivative. Such a humanized antibody comprises amino acid residue substitutions, deletions or additions in one or more non-human CDRs. The humanized antibody derivative may have substantially the same binding, better binding, or worse binding when compared to a non-derivative humanized antibody. In specific embodiments, one, two, three, four, or five amino acid residues of the CDR have been substituted, deleted or added (i.e., mutated). For further details in humanizing antibodies, see European Patent Nos. EP 239,400, EP 592,106, and EP 519,596; International Publication Nos. WO 91/09967 and WO 93/17105; U.S. Pat. Nos. 5,225,539, 5,530,101, 5,565,332, 5,585,089, 5,766,886, and 6,407,213.

As used herein, the term “hypervariable region” refers to the amino acid residues of an antibody which are responsible for antigen binding. The hypervariable region comprises amino acid residues from a “Complementarity Determining Region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., SEQUENCES OF PROTEINS OF IMMUNOLOGICAL INTEREST, 5th Ed. Public Health Service, National Institutes of Health, Bethesda, Md. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917).

As used herein, the terms “single-chain Fv” or “scFv” refer to antibody fragments comprise the VH and VL domains of antibody, wherein these domains are present in a single polypeptide chain. Generally, the Fv polypeptide further comprises a polypeptide linker between the VH and VL domains which enables the scFv to form the desired structure for antigen binding. For a review of sFv, see Pluckthun in THE PHARMACOLOGY OF MONOCLONAL ANTIBODIES, vol. 113, Rosenburg and Moore, eds. Springer-Verlag, New York, pp. 269-315 (1994). In specific embodiments, scFvs include bi-specific scFvs and humanized scFvs.

B. Methods Of Treatment

Accordingly, the present disclosure, therefore, relates to a specific binding molecule that binds to citrullinated shared epitope present on HLA-DR4 molecules, for use in treating or preventing citrulline-HLR-DR4 related autoimmune diseases, which may include, for example, inflammatory arthritis, multiple sclerosis, type 1 diabetes, lyme disease induced arthritis, rheumatoid arthritis, hydralazine-induced female systemic lupus erythematosus, pemphigoid gestationis, pemphigus foliaceus, obstructive hypertrophic cardiomyopathy, psoriatic arthritis, psoriasis, IgA nephropathy, ‘shared syndrome’-systemic sclerosis/rheumatoid arthritis and polymyalgia rheumatica. In some embodiments, a specific binding molecule that binds to citrullinated shared epitope present on HLA-DR4 molecules of the present invention, for use in treating or preventing citrulline-HLR-DR4 related autoimmune diseases, for example antibodies, are used for the treatment of rheumatoid arthritis.

In some embodiments, the diseases and conditions treatable using the methods of the present invention may include: inflammatory arthritis, rheumatoid arthritis, psoriatic arthritis, and psoriasis. The present disclosure, therefore, relates to a specific binding molecule, for example, an antibody or a specific binding fragment thereof, that specifically binds to a citrullinated shared epitope on a HLA-DR4 molecule, for use in treating or preventing diseases selected from the group consisting of arthritis, inflammatory arthritis, rheumatoid arthritis, psoriatic arthritis, and psoriasis. The present disclosure, in particular, relates to specific binding molecules (for example an antibody or specific binding fragment thereof) that have high affinity and bind specifically to a citrullinated HLA-DR4 shared epitope (for example, QKCitAA, QRCitAA, or RRCitAA) for the treatment and/or prevention of autoimmune diseases. These autoimmune diseases are wholly, or in part, mediated by the presence of citrullinated epitope QKCitAA, or citrullinated epitope QRCitAA, or citrullinated epitope RRCitAA present on HLA-DR4 receptors present on antigen presenting cells, for example, B cells. HLA-DR4 molecules bearing the citrullinated shared epitope QKCitAA, or citrullinated epitope QRCitAA, or citrullinated epitope RRCitAA presented by antigen presenting cells such as B cells are recognized by T cells as foreign thereby resulting in T-cell activation and initiation of a pathogenic immune response.

Without wishing to be bound by any specific theory, it is believed that administration of a specific binding molecule (for example an antibody or specific binding fragment thereof) reactive with a citrullinated HLA-DR4 shared epitope, can result in the binding of the specific binding molecule to antigen presenting cells, for example, B cells that present the citrullinated HLA-DR4 shared epitope to T-cells. In this way, the antibodies, or specific binding fragments thereof, of the present invention can be used to bind to the citrullinated shared epitope present on HLA-DR4 molecules and prevent the patient's T cells from reacting to the foreign looking HLA-DR4 having a citrullinated HLA-DR4 epitope. These antigen presenting cells, for example, B cells may subsequently be eliminated through antibody-dependent cellular toxicity (ADCC) mechanisms, and complement-dependent cytotoxicity (CDC) among others.

When used in the treatment of a human, the antibody, or antibody fragment thereof, preferably consists of mainly human antibody sequences, i.e. it is humanized, as to not cause the production of antibodies against the antibodies or specific binding fragments thereof of the present invention.

For therapeutic use, the antibody or a specific binding fragment thereof, suitably is stable after administered to a human patient. For example, it should have a long half-life in humans and not be broken down by proteases short time after administration. Suitable, the antibody has a half-life of weeks rather than days.

In some embodiments, the subject having a citrulline-HLR-DR4 related autoimmune disease or suspected of having a citrulline-HLR-DR4 related autoimmune disease as outlined above, may be treated with a pharmaceutical composition, comprising an effective amount of a specific binding molecule (for example, an antibody or specific binding fragment thereof which specifically binds to a citrullinated HLA-DR4 shared epitope, of the present disclosure (e.g. QKCitAA, QRCitAA, or RRCitAA), and a pharmaceutically acceptable excipient. In various embodiments, the specific binding molecule is an antibody, or fragment thereof that specifically binds to a citrullinated shared epitope e.g. QKCitAA, QRCitAA, or RRCitAA, for example, a polyclonal, or monoclonal antibody, or a chimeric antibody or a humanized antibody, or a fully human antibody that specifically binds to a citrullinated HLA-DR4 epitope, for example, a citrullinated shared epitope having the amino acid sequence QKCitAA, QRCitAA, or RRCitAA. As used herein, “pharmaceutically acceptable carrier” or “pharmaceutical acceptable excipient” includes any material which, when combined with an active ingredient, allows the ingredient to retain biological activity and is non-reactive with the subject's immune system, and can include any and all solvents, diluents, carriers, dispersion media, coatings, antibacterial and antifungal agents, isotonic and absorption delaying agents, and the like that are physiologically compatible, non-toxic, and do not interfere with the mechanism of action of the active anti-citrullinated shared epitope antibodies or antigen-binding fragments thereof. Preferably, the pharmaceutical acceptable excipient is suitable for intravenous, intramuscular, subcutaneous, parenteral, spinal or epidermal administration (e.g., by injection or infusion). Depending on the route of administration, the active component, i.e., specific binding molecule, may be coated in a material to protect the specific binding molecule from the action of acids and other proteases that may inactivate the specific binding molecule.

Formulations of the anti-citrullinated shared epitope QKCitAA QRCitAA, or RRCitAA antibody or antigen-binding fragments thereof used in accordance with the present disclosure can be prepared for storage by mixing an antibody or antigen-binding fragments thereof having the desired degree of purity with optional pharmaceutically acceptable carriers, excipients or stabilizers as amply described and illustrated in Remington's Pharmaceutical Sciences 16th edition, Osol, A. Ed. [1980], in the form of lyophilized pharmaceutically acceptable formulations, solutions and/or suspensions. Acceptable carriers, excipients, buffers or stabilizers are nontoxic to recipients at the dosages and concentrations employed, and include suitable aqueous and/or non-aqueous excipients that may be employed in the pharmaceutical compositions of the disclosure, for example, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants, buffers such as phosphate, citrate, and other organic acids. Antioxidants may be included, for example, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like; preservatives (such as octade-cyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol). Other exemplary pharmaceutically acceptable excipients may include polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In one illustrative embodiment, the pharmaceutical compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. In some embodiments, the anti-citrullinated shared epitope QKCitAA QRCitAA, or RRCitAA antibody or antigen-binding fragments thereof of the present disclosure are formulated for and can be lyophilized for storage and reconstituted in a suitable excipient prior to use according to art-known lyophilization and reconstitution techniques. In one exemplary pharmaceutical composition containing the anti-citrullinated shared epitope QKCitAA QRCitAA, or RRCitAA antibody or antigen-binding fragments thereof, the composition is formulated as a sterile, preservative-free solution of the anti-citrullinated shared epitope QKCitAA QRCitAA, or RRCitAA antibody or antigen-binding fragments thereof for intravenous or subcutaneous administration. The formulation can be supplied as either a single-use, prefilled pen, as a single-use, for example containing about 1 mL prefilled glass syringe, or as a single-use institutional use vial. Preferably, the pharmaceutical composition containing the anti-citrullinated shared epitope QKCitAA QRCitAA, or RRCitAA antibody or antigen-binding fragments thereof is clear and colorless, with a pH of about 6.9-5.0, preferably a pH of 6.5-5.0, and even more preferably a pH ranging from about 6.0 to about 5.0. In various embodiments, the formulations comprising the pharmaceutical compositions can contain from about 1,000 mg to about 10 mg, or from about 500 mg to about 20 mg, or from about 400 mg to about 30 mg or from about 300 mg to about 50 mg of the anti-citrullinated shared epitope QKCitAA QRCitAA, or RRCitAA antibody or antigen-binding fragments thereof per mL of solution when reconstituted and administered to the subject. Exemplary injection or infusion excipients can include mannitol, citric acid monohydrate, dibasic sodium phosphate dihydrate, monobasic sodium phosphate dihydrate, polysorbate 80, sodium chloride, sodium citrate and water for parenteral administration, for example, intravenously, intramuscularly, intraperitoneally, or subcutaneous administration.

In another exemplary embodiment, the anti-citrullinated shared epitope QKCitAA QRCitAA, or RRCitAA antibody or antigen-binding fragments thereof is formulated for intravenous or subcutaneous administration as a sterile aqueous solution containing from about 0.1 mg/mL to about 100 mg/mL, or more preferably, about 5-75 mg/mL, or yet more preferably, about 10-50 mg/mL, or even more preferably, about 10-40 mg/mL of antibody, with sodium acetate, polysorbate 80, and sodium chloride at a pH ranging from about 5 to 6. Preferably, the intravenous or subcutaneous formulation is a sterile aqueous solution containing 5, 10, 15, 20, 25, 30, 35, 40, 45, or 50 mg/mL of anti-citrullinated shared epitope QKCitAA antibody or antigen-binding fragments thereof, with 20 mM sodium acetate, 0.2 mg/mL polysorbate 80, and 140 mM sodium chloride at pH 5.5. Further, a solution comprising an anti-citrullinated shared epitope QKCitAA QRCitAA, or RRCitAA antibody or antigen-binding fragments thereof can comprise, among many other compounds, histidine, mannitol, sucrose, trehalose, glycine, poly(ethylene)glycol, EDTA, methionine, and any combination thereof, and many other compounds known in the relevant art.

In one embodiment, a pharmaceutical composition of the present disclosure comprises the following components: 5-100 mg anti-citrullinated shared epitope QKCitAA QRCitAA, or RRCitAA antibody or antigen-binding fragments thereof of the present disclosure, 10 mM histidine, 5% sucrose, and 0.01% polysorbate 80 at pH 5.8. This composition may be provided as a lyophilized powder. When the powder is reconstituted at full volume, the composition retains the same formulation. Alternatively, the powder may be reconstituted at half volume, in which case the composition comprises 10-200 mg anti-citrullinated shared epitope QKCitAA antibody or antigen-binding fragments thereof of the present disclosure, 20 mM histidine, 10% sucrose, and 0.02% polysorbate 80 at pH 5.8.

In one embodiment, part of the dose is administered by an intravenous bolus and the rest by infusion of the antibody formulation. For example, in some embodiments, an amount of anti-citrullinated shared epitope QKCitAA QRCitAA, or RRCitAA antibody or antigen-binding fragments thereof ranging from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg is provided via intravenous injection of the anti-citrullinated shared epitope QKCitAA QRCitAA, or RRCitAA antibody or antigen-binding fragments thereof, as a bolus, and the rest of the antibody dose may be administered by intravenous injection. A predetermined dose of the anti-citrullinated shared epitope QKCitAA antibody or antigen-binding fragments thereof, may be administered, for example, over a period of an hour to two hours to five hours.

In a further embodiment, part of the dose is administered by intravenous administration, by a subcutaneous injection and/or infusion in the form of a bolus and the rest by infusion of the antibody formulation. In some exemplary doses, the antibody formulation can be administered intravenously or subcutaneously in a dose ranging from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the anti-citrullinated shared epitope QKCitAA antibody or antigen-binding fragments thereof. In some embodiments, the dose may be given as a bolus, and the rest of the antibody dose may be administered by subcutaneous or intravenous injection. A predetermined dose of the anti-citrullinated shared epitope QKCitAA QRCitAA, or RRCitAA antibody or antigen-binding fragments thereof, may be administered, for example, over a period of an hour to two hours to five hours.

Exemplary formulations as provided herein may also contain more than one active agent as necessary for the particular indication being treated, preferably those with complementary activities that do not adversely affect each other. For example, it may be desirable to provide antibodies with other specificities. Alternatively, or in addition, the composition may comprise an anti-inflammatory agent, a chemotherapeutic agent, a cytotoxic agent, a cytokine, a growth inhibitory agent and/or a small molecule enzyme inhibitor or receptor modulator. Such molecules are suitably present in combination in amounts that are effective for the purpose intended.

The formulations to be used for in vivo administration should be sterile, or nearly so. This is readily accomplished by filtration through sterile filtration membranes.

In various embodiments, illustrative formulations of the pharmaceutical compositions described herein can be prepared using methods widely known in the field of pharmaceutical formulations. In general, such preparatory methods can include the step of bringing the active ingredient into association with a carrier or one or more other accessory ingredients, and then, if desirable, packaging the product into a desired single-or multi-dose unit.

In some embodiments, the pharmaceutical composition can be also delivered in a vesicle, in particular, a liposome containing one or more liposomal surface moieties for example, polyethylene glycol, antibodies and antibody fragments thereof, which are selectively transported into specific cells or organs, thus enhance targeted drug delivery.

The optimum concentration of the active ingredient(s) in the chosen medium can be determined empirically, according to procedures well known to the skilled artisan, and will depend on the ultimate pharmaceutical formulation desired and the use to be employed.

The present disclosure also provides a pharmaceutical pack or kit comprising one or more containers filled with one or more of the ingredients of the pharmaceutical compositions of the disclosure, which at minimum will include an anti-citrullinated shared epitope QKCitAA, QRCitAA, or RRCitAA antibody or an antigen-binding fragment thereof as described herein. In other embodiments, the kit may contain one or more further containers providing a pharmaceutically acceptable excipient, for example a diluent. In one embodiment, a kit may comprise at least one container, wherein the container can include an anti-citrullinated shared epitope QKCitAA QRCitAA, or RRCitAA antibody or antigen-binding fragments thereof of the present disclosure, or a pharmaceutical composition disclosed herein and/or an anti-inflammatory drug, and/or an immunoconjugate comprising a cytotoxin, and/or a chemotherapeutic drug, and/or an immunosuppressant and/or a radioisotope. The kit may also include a set of instructions for preparing and administering the final pharmaceutical composition to the subject in need thereof, for the treatment of a citrulline-HLR-DR4 mediated disease or disorder.

In various embodiments, the appropriate dose of the active agents described herein is made by the clinician, e.g., using parameters or factors known or suspected in the art to affect treatment or predicted to affect treatment. In some embodiments, sound medical practice will dictate that the initial dose begins with an amount somewhat less than the optimum dose and it is increased by small increments thereafter until the desired or optimum effect is achieved relative to any negative side effects. Important diagnostic measures include those symptoms of, e.g., the inflammation or level of inflammatory cytokines produced, autoantibody titers, tissue damage, or estimated activity or stage in a citrulline-HLR-DR4 mediated disease course, for example, rheumatoid arthritis. In some embodiments, the actual dosage levels of the active ingredients in the pharmaceutical compositions of the present disclosure may be varied so as to obtain an amount of the active ingredient which is effective to achieve the desired therapeutic response for a particular subject, composition, and mode of administration, without being toxic to the patient. Compositions comprising anti-citrullinated shared epitope QKCitAA QRCitAA, or RRCitAA antibody or antigen-binding fragments thereof, of the present disclosure can be administered to the subject, for example, a human subject by one or more administration modalities, for example, by intravenous injection, continuous infusion, or by doses at intervals of, e.g., one day, several days, one week, or 1-7 times per week. Doses may be provided parenterally, for example, intravenously, or subcutaneously.

By way of illustration only, and taking into consideration various factors for determining appropriate doses and dosing frequencies, an exemplary dose comprising an anti-citrullinated shared epitope QKCitAA QRCitAA, or RRCitAA antibody or antigen-binding fragments thereof to be administered to a patient in need thereof can include a single dose of about 0.01 to about 100 mg/kg body weight, more preferably about 0.02 to about 50, more preferably about 0.02 to about 10, more preferably about 0.02 to about 7, about 0.03 to about 5, or about 0.05 to about 3 mg/kg body weight dosed, once or more times per day, and/or one or more times per week, for example, for one to four weeks, or one to eight weeks, or one to twelve weeks, or one to fourteen weeks, or for a period ranging from one month to several years as therapeutically required. In some embodiments, an exemplary dosing regimen can include administration of a maximal dose or dosing frequency that avoids significant undesirable side effects. In some embodiments, a total daily or weekly dose may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 0.5 mg/kg, at least 1.0 mg/kg, at least 2.0 mg/kg, at least 10 mg/kg, at least 15 mg/kg, at least 20 mg/kg, at least 25 mg/kg, or at least 50 mg/kg, or at least or at least 100 mg/kg.

In another example, an illustrative dose (which may be a therapeutically effective dose, or a therapeutic dose) comprising an anti-citrullinated shared epitope QKCitAA, QRCitAA, or RRCitAA antibody or antigen-binding fragment thereof of the disclosure, to be administered to a patient in need thereof may be about 0.001 mg/kg to about 200 mg/kg of the patient's body weight dosed per day, administered in a single dose, eg. a unit dose, or divided doses administered two or times per day. The dosage to a subject in need thereof, may be between 0.001 mg/kg and 200 mg/kg, 0.001 mg/kg and 100 mg/kg, 0.001 mg/kg and 50 mg/kg, 0.001 mg/kg and 25 mg/kg, 0.001 mg/kg and 10 mg/kg, 0.001 mg/kg and 5 mg/kg, 0.001 mg/kg and 1 mg/kg 0.001 mg/kg and 0.5 mg/kg and any dosage amount there between. As non-limiting examples, treatment according to the present disclosure may be provided as a daily dosage of an antibody or an antigen binding fragment thereof, in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof

Depending on the severity of the condition, and the various factors discussed herein, the dose, frequency and the duration of the treatment can be adjusted accordingly, in view of proper medical standards known to those of skill in the art. In certain exemplary embodiments, the anti-citrullinated shared epitope QKCitAA QRCitAA, or RRCitAA antibody or antigen-binding fragment thereof of the disclosure can be administered as an initial dose of at least about 0.1 mg to about 800 mg, about 1 to about 500 mg, about 5 to about 300 mg, or about 10 to about 200 mg, to about 100 mg, or to about 50 mg. The first dose may be an initial loading dose, to be followed subsequently by a plurality of maintenance doses. In certain exemplary embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks, or doses of anti-citrullinated shared epitope QKCitAA QRCitAA, or RRCitAA antibody or antigen-binding fragment thereof of the disclosure may be repeated and the administrations may be separated by at least 1 day, 2 days, 3 days, 5 days, 10 days, 15 days, 30 days, 45 days, 2 months, 75 days, 3 months, or at least 6 months.

The route of administration of the compositions of the present disclosure may be by, e.g., topical or cutaneous application, injection or infusion by intravenous, intraperitoneal, subcutaneous, intracerebral, intramuscular, intraocular, intraarterial, intradermal, intracerebrospinal, intralesional, subcuticular, intraarticular, subcapsular, subarachnoid, intraspinal, epidural and intrasternal injection and infusion, or by sustained release systems or an implant. The injectable preparations may include dosage forms for intravenous, subcutaneous, intracutaneous and intramuscular injections, drip infusions, etc. Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DIS-ETRONIC™ pen (Disetronic Medical Systems, Burghdorf, Switzerland), HUMALOG MIX 75/25™ pen, HUMA-LOG™ pen, HUMALIN 70/30™ pen (Eli Lilly and Co., Indianapolis, Ind.), NOVOPENTM I, II and III (Novo Nordisk, Copenhagen, Denmark), NOVOPEN JUNIOR™ (Novo Nor-disk, Copenhagen, Denmark), BD™ pen (Becton Dickinson, Franklin Lakes, N.J.), OPTIPEN™, OPTIPEN PRO™ OPTIPEN STARLET™, and OPTICLIK™ (Sanofi-Aventis, Frankfurt, Germany), as exemplary pen based delivery methods contemplated herein in the administration of the present anti-citrullinated shared epitope QKCitAA antibody or antigen-binding fragment thereof. Illustrative examples of pen based devices having applications in subcutaneous delivery of a pharmaceutical composition of the present disclosure include, the SOLOSTAR™ pen (Sanofi-Aventis), the FLEXPEN™ (Novo Nordisk), and the KWIKPEN™ (Eli Lilly).

In various embodiment, the anti-citrullinated shared epitope QKCitAA QRCitAA, or RRCitAA antibody or antigen-binding fragment thereof according to the invention can be administered to a patient exhibiting one or more arthritis criteria in accordance with sound medical practice, for example, when a subject is deemed to have rheumatoid arthritis in view of the 2010 ACR/EULAR RA Classification Criteria, which are incorporated herein by reference in its entirety. In some embodiments, subjects who present with undifferentiated inflammatory arthritis, or undifferentiated synovitis, or are found to have definite RA in accordance with the 2010 American College of Rheumatology/European League Against Rheumatism classification criteria for rheumatoid arthritis as shown herein in Table 1 may be treated with the anti-citrullinated shared epitope QKCitAA QRCitAA, or RRCitAA antibody or antigen-binding fragment thereof according to the invention. In various embodiments, treatment by administration of the anti-citrullinated shared epitope QKCitAA QRCitAA, or RRCitAA antibody or antigen-binding fragment thereof according to the invention may commence at any time, and continued until symptoms resolve or for a predetermined period of time as indicated by the remission of symptoms or for an indefinite period of time.

TABLE 1
The 2010 American College of Rheumatology/European League Against
Rheumatism classification criteria for rheumatoid arthritis

| | Score |
|---|---|
| Target population (Who should be tested?): Patients who | |
| 1) have at least 1 joint with definite clinical synovitis (swelling)* | |
| 2) with the synovitis not better explained by another disease† | |
| Classification criteria for RA (score-based algorithm: add score of categories A-D; | |
| a score of ~6/10 is needed for classification of a patient as having definite RA)‡ | |
| A. Joint involvement§ | |
| 1 large joint¶ | 0 |
| 2-10 large joints | 1 |
| 1-3 small joints (with or without involvement of large joints)# | 2 |
| 4-10 small joints (with or without involvement of large joints) | 3 |
| >10 joints (at least 1 small joint)** | 5 |
| B. Serology (at least 1 test result is needed for classification)†† | |
| Negative RF and negative ACPA | 0 |
| Low-positive RF or low-positive ACPA | 2 |
| High-positive RF or high-positive ACPA | 3 |
| C. Acute-phase reactants (at least 1 test result is needed for classification)‡‡ | |
| Normal CRP and normal ESR | 0 |
| Abnormal CRP or abnormal ESR | 1 |
| D. Duration of symptoms§§ | |
| <6 weeks | 0 |
| ~6 weeks | 1 |

*The criteria are aimed at classification of newly presenting patients. In addition, patients with erosive disease typical of rheumatoid arthritis (RA) with a history compatible with prior fulfillment of the 2010 criteria should be classified as having RA. Patients with longstanding disease, including those whose disease is inactive (with or without treatment) who, based on retrospectively available data, have previously fulfilled the 2010 criteria should be classified as having RA.
†Differential diagnoses vary among patients with different presentations, but may include conditions such as systemic lupus erythematosus, psoriatic arthritis, and gout. If it is unclear about the relevant differential diagnoses to consider, an expert rheumatologist should be consulted.
‡Although patients with a score of <6/10 are not classifiable as having RA, their status can be reassessed and the criteria might be fulfilled cumulatively over time.
§Joint involvement refers to any swollen or tender joint on examination, which may be confirmed by imaging evidence of synovitis. Distal interphalangeal joints, first carpometacarpal joints, and first metatarsophalangeal joints are excluded from assessment. Categories of joint distribution are classified according to the location and number of involved joints, with placement into the highest category possible based on the pattern of joint involvement.
¶“Large joints” refers to shoulders, elbows, hips, knees, and ankles.
#“Small joints” refers to the metacarpophalangeal joints, proximal interphalangeal joints, second through fifth metatarsophalangeal joints, thumb interphalangeal joints, and wrists.
**In this category, at least 1 of the involved joints must be a small joint; the other joints can include any combination of large and additional small joints, as well as other joints not specifically listed elsewhere (e.g., temporomandibular, acromioclavicular, stemoclavicular, etc.).
††Negative refers to IU values that are less than or equal to the upper limit of normal (ULN) for the laboratory and assay; low-positive refers to IU values that are higher than the ULN but 3 times the ULN for the laboratory and assay; high-positive refers to IU values that are >3 times the ULN for the laboratory and assay. Where rheumatoid factor (RF) information is only available as positive or negative, a positive result should be scored as low-positive for RF. ACPA = anti-citrullinated protein antibody.
‡‡Normal/abnormal is determined by local laboratory standards.
CRP = C-reactive protein;
ESR = erythrocyte sedimentation rate.
§§Duration of symptoms refers to patient self-report of the duration of signs or symptoms of synovitis (e.g., pain, swelling, tenderness) of joints that are clinically involved at the time of assessment, regardless of treatment status.

The anti-citrullinated shared epitope QKCitAA QRCitAA, or RRCitAA antibody or antigen-binding fragment thereof according to the invention can be used in diagnosis or as a research tool. For example, one or more antibodies according to the invention may be included as positive controls in a diagnostic kit for testing for the presence of antigen presenting cells, bearing citrullinated HLA-DR4 shared epitope, the causative agent in promoting T cell mediated immune activity, including upregulation of cytokine production (e.g. TNF-α, IL-6 and IL-17) T-cell proliferation and recruitment of other pro-inflammatory cells.

Suitable concentrations for the antibodies or specific binding fragments thereof when used in vitro can be from 10 ng/mL to 100 μg/mL. The appropriate concentration which yields a suitable signal with low background (good signal to noise ratio) can be found by a person skilled in the art. Suitable medium for the dilution of the antibodies are also known in the art and can, for example, be phosphate buffered saline optionally with a supplement of BSA.

One further aspect of the invention is a diagnostic kit that comprises an antibody according to the invention. Such a kit preferably comprises an ELISA plate, flow cytometry, or other platform for antibody analysis as well as reagents for detection of antibodies, such as labeled-anti-human antibodies and suitable buffers. Thus, the antibodies according to the invention can be used for in vitro diagnosis. In some embodiments, the subject may be initially screened for the presence of citrullinated epitope QKCitAA, QRCitAA, or RRCitAA presented on the surface of the patient's white blood cells. This diagnostic test can be performed using an anti-citrullinated shared epitope QKCitAA QRCitAA, or RRCitAA antibody or antigen-binding fragment thereof according to the invention. In some embodiments, the anti-citrullinated shared epitope QKCitAA QRCitAA, or RRCitAA antibody or antigen-binding fragment thereof is labeled with a detection moiety, for example, a fluorescence protein or agent (e.g. fluorescein, or GFP), a radioactive label, a chemiluminescent label etc. The presence of a citrullinated shared epitope QKCitAA QRCitAA, or RRCitAA on the surface of the patient's white blood cells, for example, an antigen presenting cell (for example a B cell) indicates that the subject having an autoimmune disease as defined herein may be treatable using an anti-citrullinated shared epitope QKCitAA QRCitAA, or RRCitAA antibody or antigen-binding fragment thereof

In some embodiments, subjects who may not be confirmed as having RA but may be suitable candidates for treatment can have one or more diagnostic tests performed to determine whether they carry citrullinated shared epitope QKCitAA QRCitAA, or RRCitAA. In some embodiments, a diagnostic test would be used to detect the presence of the citrullinated shared epitope on peripheral blood B cells. Several assays could be used, for example, 1) Flow cytometry using the anti-Cit SE mAb; 2) An ELISA of cell lysates using the same mAb as the capture antibody in a sandwich (e.g. IF9 monoclonal anti- QKCitAA SE HLA-DR4 antibody, and a second anti-DR4 mAb that recognizes a non-citrullinated native epitope; and 3) A Mass spectrometry analysis of immunoprecipitated HLA-DR4 from B cell lysates from the subject being tested. The immunoprecipitation could use any anti-HLA-DR4 mAb (for example, anti- DR4 mAb (L243)). The immunoprecipitated material could then be subjected to trypsin digestion and the resulting peptides could be resolved and identified using LC-mass spectrometry or MS, since the citrullinated epitope peptide would have a mass that is expected to be larger than if one of the arginine residues in the epitopes QKRAA QRRAA, or RRRAA were not citrullinated. In some embodiments, a diagnostic assay can be used to identify a subject which may benefit from treatment using a pharmaceutical composition comprising an anti-citrullinated shared epitope QKCitAA QRCitAA, or RRCitAA antibody or antigen-binding fragment thereof. In the instant diagnostic assay, molecular imaging of the rheumatoid synovium in RA patients may be performed using the anti-citrullinated shared epitope QKCitAA QRCitAA, or RRCitAA antibody or antigen-binding fragment thereof of the present invention labeled with ^{99m}Tc for single photon emission computed tomography (SPECT) imaging, or labeled with a near-infrared dye (NIR) of optical imaging.

### EXAMPLES

### Example 1

### Production of Monoclonal Antibodies To Citrullinated EQKCitAA Peptide Hapten

Immunization of mice

A peptide with the amino acid sequence C-aminohexanoic acid-EQKCitAA was designed by scientists at Cayman Chemical and its synthesis was contracted to LifeTein, LLC. The N terminal cysteine (C) was incorporated to allow chemical linkage to a carrier protein. The aminohexanoic acid is a linker that provides physical space between the carrier protein and the desired hapten. A total of six amino acids from the DRB1*0401, amino acids 69-74 (EQKRAA) were included as the hapten, with the arginine (R) normally found at position 72 replaced by the amino acid citrulline (Cit). This peptide was coupled to keyhole limpet hemocyanin (KLH) carrier protein using a commercial kit (Imject Maleimide Activated Carrier Protein Spin Kit, Thermo Scientific) to produce the “immunogen”.

The immunogen was diluted to a concentration of 200 μg/ml in phosphate buffered saline (PBS). On Day 0, 1 ml of the immunogen was mixed with 1 ml of Complete Freund's Adjuvant (CFA) and emulsified. Three BALB/c female mice each were immunized intraperitoneally (IP) with 50 μg of the immunogen:CFA emulsion. On Days 14 and 34, 1 ml of the immunogen was mixed with 1 ml of Incomplete Freund's Adjuvant (IFA) and emulsified. The three BALB/c female mice each were injected again (boosted) IP with 50 μg of the immunogen:IFA emulsion. On Day 65, 50 μg of the immunogen was injected into the tail veins of the mice without adjuvant. On Day 68, one mouse was sacrificed and the spleen harvested for hybridoma production.

Production of Monoclonal Antibodies

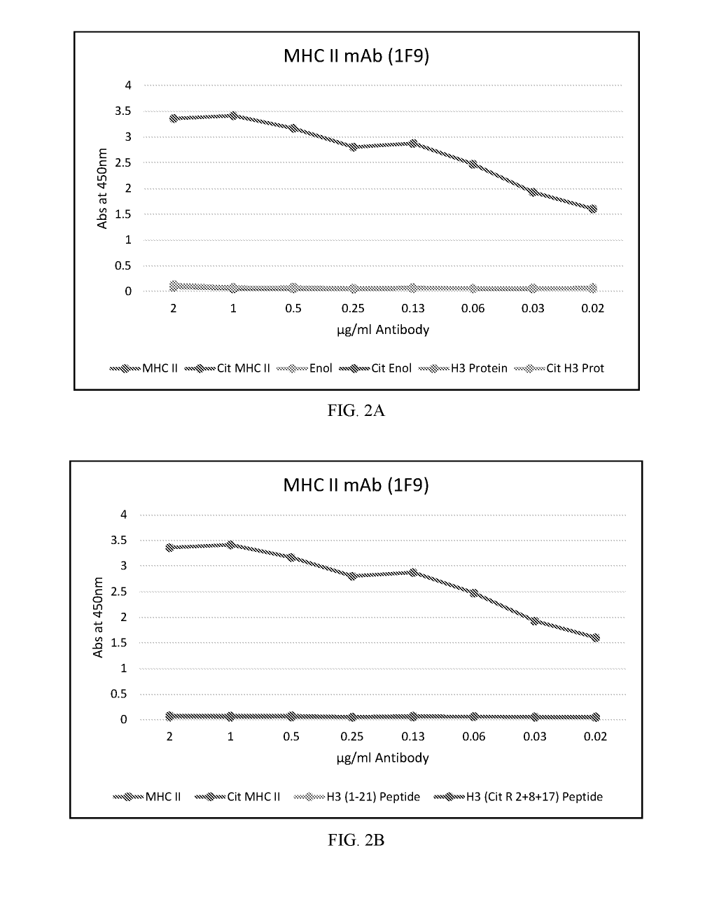

A sterile single cell suspension was made from the mouse spleen cells in RPMI-1640 basal medium. The red blood cells in the spleen were lysed by treatment with TRIS-buffered ammonium chloride. The remaining white blood cells were washed in RPMI-1640, resuspended in RPMI-1640, and combined with P3x63Ag8.653 myeloma cells in a 5:1 spleen cell/myeloma ratio. The cells were pelleted, the liquid aspirated, and the cell pellet treated with 50% polyethylene glycol to induce cell fusion and hybridoma formation. The fused cells were seeded into 96-well tissue culture plates and grown in HAT medium to select for hybridomas. After 10 days of selection, the supernatants were tested for the presence of monoclonal antibodies capable of binding to the peptide portion of the immunogen. This screening was performed by ELISA against plate-bound hapten peptide (C-aminohexanoic acid-EQKCitAA). Similarly, counter-screening to determine selectivity was performed by ELISA against plate-bound control peptide (C-aminohexanoic acid-EQKRAA) containing an arginine instead of a citrulline. One monoclonal antibody, 1F9, was found to bind to the citrulline-containing peptide, but not the arginine containing peptide. This monoclonal antibody was further characterized by ELISA for binding to plate bound human alpha enolase, citrullinated human alpha enolase, histone H3 (full length protein), citrullinated histone H3 (full length protein), histone H3 tail peptide (amino acids 1-21), and citrullinated histone H3 tail peptide (citrullinated at R2, R8, and R17). The 1F9 antibody bound only the citrullinated hapten peptide (C-aminohexanoic acid-EQKCitAA). It did not bind any other peptide or protein tested, citrullinated or not. This indicates that the 1F9 monoclonal antibody is specific for the citrullinated form of the shared epitope sequence. To support the finding of specific binding of 1F9 monoclonal antibody is specific for the citrullinated form of the shared epitope sequence, various antigens were plated in 96 well plates and screened using various concentrations of 1F9 monoclonal antibody. The antigens used to coat the plates included MHC class II peptide (EQKRAA), citrullinated MHC class II peptide (EQKCitAA), enolase, citrullinated enolase, H3 protein and citrullinated H3 protein. As shown in FIG. 2A and 2B, 1F9 monoclonal antibody binds to the citrullinated peptide specifically. Even when peptides were used to test the specificity of 1F9 monoclonal antibody, for example, H3 (1-21) peptide and citrullinated H3 peptide (1-21) (Cit Arginine at positions 2, 8 and 17), only citrullinated MHC class II peptide bound specifically to the IF9 monoclonal antibody.

### Example 2

### Citrullination of DRB1*0401 Protein on B cells From HLA-DR4 Trans2enic Mice in vitro With Recombinant PAD4

Mice expressing the human HLA-DRB1*0401 gene in the absence of endogenous mouse MHC Class II expression are obtained from Taconic, model # 4149-F or 4149-M. B cells from the spleens of these mice express the HLA-DR4 heterodimeric protein on their surface, including the DRB1*0401 beta chain containing the shared epitope amino acid sequence QKRAA. A spleen from one of these mice is harvested under sterile conditions, and a single cell suspension is produced as described in Current Protocols in Immunology, Unit 3.1, “Isolation of Mouse Mononuclear Cells”, the disclosure of which is incorporated herein by reference in its entirety. The spleen cells are suspended in TRIS-buffered saline solution supplemented with 1 mM CaCl₂ at 1×10⁷ cells/ml.

Aliquots of 0.5 mL of spleen cells are treated with recombinant human PAD4 (Cayman Chemical, Catalog No. #10500, Ann Arbor, Mich. USA) at 0, 0.05, 0.2, 1, and 5 μg per mL for 30 minutes at 37° C. The cells are centrifuged at 150×g for 5 minutes, the supernatant discarded, and the cell pellet resuspended in 0.2 ml FACS buffer (Hanks Balanced Salt Solution supplemented with 0.1% bovine serum albumin and 0.01% sodium azide).

To the cell suspensions is added 2 μL of FITC-conjugated anti-B220 mAb (BioLegend catalog #103205) and 2 micrograms of APC-conjugated 1F9 mAb. Anti-B200 stains all B cells and the 1F9 mAb stains only B cells expressing the citrullinated shared epitope. The cells are incubated 30 minutes on ice, centrifuged at 150×g, and the pellets resuspended in FACS buffer. The cells are analyzed by flow cytometry. PAD4 treatment results in the citrullination of the shared epitope region of the DRB1*0401 chain that is subsequently recognized by the 1F9 antibody.

### Example 3

### Citrullination of DRB1*0401 Protein On B cells from HLA-DR4 Transgenic Mice Induces T cell Proliferation and Cytokine Production

Recombinant human PAD4 (Cayman Chemical, Catalog No. #10500, Ann Arbor, Mich. USA) is chemically coupled to goat anti-mouse IgG/IgM/IgA antibody (ThermoFisher catalog No. A-10666) in a 1:1 molar ratio using the S-HyNic/4FB conjugation technology from Solulink (Solulink Inc., Catalog No. S-1002, San Diego, Calif. USA) following manufacturer's instructions. Essentially, the recombinant human PAD4 is conjugated to the S-HyNic linker (succinimidyl-6-hydrazino-nicotinamide) and is conjugated to the goat anti-mouse IgG/IgM/IgA antibody via a 4FB (4-formylbenzamide) linker coupled to the goat anti-mouse IgG/IgM/IgA antibody. The HyNic-modified recombinant human PAD4 is incubated with 4FB-modified goat anti-mouse IgG/IgM/IgA antibody in a TurboLink™ catalyzed conjugation reaction in accordance with the manufacturer's protocol (Solulink Inc., Catalog No. S-2006-105, San Diego, Calif. USA).

The result is recombinant human PAD4/goat anti-mouse IgG/IgM/IgA antibody complex conjugated through a UV-traceable, stable bond (bis-arylhydrazone) with measurable absorbance at 354 nm. This “PAD⁴/ₐnti-mouse Ig conjugate” is diluted to a concentration of 1 mg/mL in complete medium, sterile filtered, and stored at 4° C.

Mice expressing the human HLA-DRB1*0401 gene in the absence of endogenous mouse MHC Class II expression are obtained from Taconic, (Abb Knockout/Transgenic HLA-DR4 Catalog No. 4149-F or 4149-M Taconic Biosciences Rensselaer, N.Y. USA). B cells from the spleens of these mice express the HLA-DR4 heterodimeric protein on their surface, including the DRB1*0401 beta chain containing the shared epitope sequence QKRAA. A spleen from one of these mice is harvested under sterile conditions, and a single cell suspension is produced as described in Current Protocols in Immunology, Unit 3.1, “Isolation of Mouse Mononuclear Cells”, the disclosure of which is incorporated herein by reference in its entirety. The spleen cells are suspended in “complete medium” (RPMI-1640 supplemented with 10% fetal calf serum, 1 mM CaCl₂, and antibiotics) at a concentration of 2×10⁶/mL and chilled to 4° C. on ice for 30 minutes.

10 ml (2×10⁷ total cells) of the spleen cell suspension is centrifuged at 150×g for 5 minutes at 4° C., the supernatant discarded, and the cell pellet is resuspended in 1 mL of 4° C. complete medium. To this 1 mL of the spleen cell suspension, 1004 of the sterile PAD⁴/ₐnti-mouse Ig conjugate is added and incubated on ice for 1 hour. The cells are then centrifuged at 150×g for 5 minutes at 4° C., the supernatant discarded, and the cell pellet is resuspended in 10 ml of 4° C. complete medium. The cells are again centrifuged at 150×g for 5 minutes at 4° C., the supernatant discarded, and the cell pellet is resuspended in 10 mL of 4° C. complete medium. The surface immunoglobulin receptors on the B cells in this suspension are now bound by the anti-mouse Ig/PAD4 conjugate and unbound conjugate has been washed away.

The 1F9 monoclonal antibody that binds to the citrullinated shared epitope is diluted in complete medium at 1 mg/mL, sterile filtered, and kept at 4° C. until ready to use.

In a sterile 96-well round-bottom microtiter plate, the spleen cells and antibody are mixed and cultured in the following conditions:

A. Six wells containing 100 μL of the spleen cell suspension (2×10⁵ cells) that are not treated with PAD4/anti-mouse Ig conjugate plus 100 μL of complete medium.

B. Six wells containing 100 μL of the spleen cell suspension (2×10⁵ cells) that are treated with PAD4/anti-mouse Ig conjugate plus 100 μL of complete medium.

C. Six wells containing 100 μL of the spleen cell suspension (2×10⁵ cells) that are not treated with PAD4/anti-mouse Ig conjugate plus 100 μL of 1F9 monoclonal antibody dilution.

D. Six wells containing 100 μL of the spleen cell suspension (2×10⁵ cells) that are treated with PAD4/anti-mouse Ig conjugate plus 100 μL of 1F9 monoclonal antibody dilution.

The 96-well plate is transferred to a 37° C. CO₂ incubator for 72 hours. After 72 hours of culture, the supernatants for three of the six wells at each condition are removed and assessed for production of TNF-α by ELISA (See for example, TNF-α (mouse) ELISA Kit Catalog No. 500850 Cayman Chemical, Ann Arbor, Mich. USA) following manufacturer's instructions. For the remaining three wells at each condition, the degree of T cell proliferation in each well is assessed by ³H-thymidine incorporation as described in Current Protocols in Immunology, Unit 7.10.16, Support Protocol 2, the disclosure of which is incorporated herein by reference in its entirety.

No TNF-α production or T cell proliferation are detected under conditions A or C. The highest levels of TNF-α production and T cell proliferation are detected under condition B. Intermediate levels of TNF-α and T cell proliferation are detected under condition D.

### Example 4

### Identification of Citrullinated HLA-DRB1*0401 On B Cells Isolated From RA Patients

Freshly drawn peripheral blood obtained from human patients with anti-citrullinated protein antibody positive (ACPA+) rheumatoid arthritis and healthy controls are purchased from Dx Biosamples (San Diego Calif. USA) (or other qualified vendor). Two mLs of each blood sample are added to 20 ml ACK lysing buffer (Thermo Fisher catalog #A1049201) for 10 minutes at room temperature to lyse the red blood cells. The samples are then centrifuged at 150×g for 5 minutes, and the cell pellet containing living leukocytes is resuspended in 2 mL of lysis buffer (50 mM Tris, 150 mM NaCl, 5 mM EDTA, 0.5% NP-40, 10 mM iodoacetamide, and protease inhibitors (Roche)). The lysate is centrifuged at 10,000×g for 30 minutes to remove insoluble material. The cleared lysate is moved to a new tube and incubated (precleared) with 50 μL of sepharose beads for 30 minutes at 4° C. The lysates are centrifuged for 10 minutes at 10,000×g to pellet the sepharose. The precleared lysate is transferred to a new tube and 50 μL of sepharose beads covalently coupled to 100 μg of a mouse anti-human HLA-DR [L243] monoclonal antibody (Abcam, Anti-HLA DR antibody [L243], Catalog No. ab136320, Cambridge, Mass. USA) is added to the lysate and incubated for 1 hour with rotation. The lysate and L243-sepharose are incubated at 4° C. for 4 hours with rotation. The tubes are centrifuged for 10 minutes at 1,000×g in a tabletop microfuge, and the supernatant discarded from the pelleted L243-sepharose conjugate. The L243-sepharose conjugate is washed with 1 mL of lysis buffer followed by washing with 1 mL of a wash solution containing 20 mM Tris-HCl, 120 mM NaCl, pH8.0. The HLA-DR molecules are eluted from the L243-sepharose conjugate by the addition of 0.5 mL of 0.1 M glycine-HC1, pH 3.0. The eluted HLA-DR4 molecules are sent to MS Bioworks, (Ann Arbor Mich., USA) for tryptic digest amino acid analysis to determine if there is citrullination of the shared epitope region QKRAA.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

What is claimed is:

1. A method for treating an autoimmune disease in a human subject mediated by the presence of a HLA-DR4 citrullinated shared epitope comprising an amino acid sequence: QKCitAA, QRCitAA, or RRCitAA, the method comprising administering a therapeutically effective amount of an antibody or a specific binding fragment thereof that specifically binds to citrullinated epitope QKCitAA, QRCitAA, or RRCitAA to the human subject in need thereof.

2. The method according to claim 1, wherein the autoimmune disease is inflammatory arthritis, multiple sclerosis, type 1 diabetes, lyme disease induced arthritis, rheumatoid arthritis, hydralazine-induced female systemic lupus erythematosus, pemphigoid gestationis, pemphigus foliaceus, obstructive hypertrophic cardiomyopathy, psoriatic arthritis, psoriasis, IgA nephropathy, ‘shared syndrome’-systemic sclerosis/rheumatoid arthritis and polymyalgia rheumatic.

3. The method according to claim 2, wherein the autoimmune disease is multiple sclerosis, rheumatoid arthritis, type 1 diabetes, and Lyme disease induced arthritis.

4. The method according to claim 3, wherein the autoimmune disease is rheumatoid arthritis.

5. The method according to claim 1, wherein the subject is initially screened for the presence of citrullinated epitope QKCitAA, QRCitAA, or RRCitAA present on the surface of the patient's white blood cells.

6. The method according to claim 5, wherein the presence of citrullinated epitope QKCitAA, QRCitAA, or RRCitAA is determined using the patient's B cells.

7. The method according to claim 6, wherein the presence of citrullinated epitope QKCitAA on the patient's B cells is determined by admixing the patient's B cells with the monoclonal antibody IF9, and measuring the specific binding between monoclonal antibody IF9 and the citrullinated epitope QKCitAA.

8. The method according to claim 7, wherein the presence of citrullinated epitope QKCitAA on the patient's B cells is determined using flow cytometry.

9. The method according to claim 1, wherein the antibody or a specific binding fragment thereof that specifically binds to citrullinated epitope QKCitAA, QRCitAA, or RRCitAA is administered intravenously, subcutaneously or intraperitoneally.

10. The method according to claim 1, wherein the patient is also administered an anti-inflammatory agent, a chemotherapeutic agent, a cytotoxic agent, a cytokine, a growth inhibitory agent, a small molecule enzyme inhibitor or receptor modulator or a combination thereof

11. An antibody or a specific binding fragment thereof, that specifically binds to a human citrullinated epitope QKCitAA, QRCitAA, or RRCitAA present on a HLA-DR4 .

12. The antibody or a specific binding fragment thereof according to claim 11, that specifically binds to a human citrullinated epitope QKCitAA.

13. The antibody or a specific binding fragment thereof according to claim 11, selected from the group consisting of a monoclonal antibody, a polyclonal antibody, a chimeric antibody, a humanized antibody, and a wholly human antibody.

14. The antibody or a specific binding fragment thereof according to claim 13, wherein the antibody or a specific binding fragment thereof which specifically binds to a human citrullinated epitope QKCitAA designated as monoclonal antibody IF9, or an antigen binding fragment thereof.
